(19) United States
(12) Patent Application Publication
(10) Pub. No.: US 2011/0178316 A1
(43) Pub. Date: Jul. 21, 2011

(54) NOVEL FULLERENE CYCLOPROPANATION REACTION

(75) Inventor: Jing Zhang, Danville, VA (US)

(73) Assignee: Luna Innovations Incorporated, Roanoke, VA (US)

(21) Appl. No.: 13/056,405

(22) PCT Filed: Jul. 28, 2009
(86) PCT No.: PCT/US09/04358

§ 371 Date: Mar. 25, 2011

Related U.S. Application Data
(60) Provisional application No. 61/129,925, filed on Jul. 30, 2008.

(51) Int. Cl. C07C 247/10; C07C 265/08; C07C 211/31; C07C 229/36; C07C 271/12; C07C 275/24

(52) U.S. Cl. 552/6; 560/355; 564/387; 560/38; 562/443; 560/28; 564/56

(57) ABSTRACT

Fullerene compounds represented by the formulae Cm[C(R)CON3]n, Cm[C(R)NCO]n, Cm[C(R)NHCORd]n, and Cm[C(R)NH2]n, wherein Cm represents a fullerene moiety having m carbon atoms, m represents an even integer from about 60 to about 200, n represents an integer of about 1 or more, R represents an electron-withdrawing group, and Rd represents an alkoxy or alkylamino group. In addition, processes for preparing these fullerene compounds involving reacting an acyl azide with a fullerene in the presence of a base.

### FIELD

This disclosure relates to fullerene cyclopropanation reactions and preparation of fullerenyl amino acids.

### BACKGROUND

Fullerenes are a third allotrope of carbon. It is desirable to prepare fullerene derivatives containing functional groups which can have applications in the field of research and/or have utility as building blocks for other useful materials, which may possess improved physical properties, such as solubility or polarity, of the fullerene derivatives.

Methanofullerenes are a class of extensively studied fullerene derivatives because of their synthetic availability and the unique characteristic properties of fullerene.

Several methods have been developed for the preparation of methanofullerenes. For example, Wudl et al. reported reactions of fullerenes in 1,3-dipolar cycloadditions with diazo compounds. Wudl et al., The Chemical Properties of Buckminsterfullerene (C₆₀) and the Birth and Infancy of Fulleroids, Acc. Chem. Res. 1992, 25, 157-61. In addition, Vasella et al, described reactions of fullerenes in [2+1] carbene additions with nucleophilic glycosylidenecarbenes. Vasella et al., Herstellung enantiomeren—reiner, spiroverknüpfier C-Glycoside von C60. Glycosylidencarbene Teil 11, Angew. Chem., 1992, 104, 1383-85. Further, Hirsch et al. reported addition of nucleophiles such as organolithium and organomagnesium compounds. Hirsch et al., Titration von C₆₀: eine Methode zur Synthese von Organofullerene, Angew. Chem. 1992, 104, 808-10. Moreover, Bingel reported cycloaddition reactions of fullerenes and specific 1,3-dicarbonyl compounds. See U.S. Pat. No. 5,739,376.

### SUMMARY

An exemplary embodiment is directed to a fullerene compound represented by the formula (I): Cₘ[C(R)CON₃]ₙ,

wherein Cₘ represents a fullerene moiety having m carbon atoms,

m represents an even integer from about 60 to about 200,

n represents an integer of about 1 or more, and

R represents an electron-withdrawing group.

Another exemplary embodiment is directed to a fullerene compound represented by the formula (II): Cₘ[C(R)NCO]ₙ, wherein Cₘ, m, n, and R have the same meaning as defined above.

A further exemplary embodiment is directed to a fullerene compound represented by the formula (III): Cₘ[C(R)NHCOR_{d}]ₙ, wherein Cₘ, m, n, and R have the same meaning as defined above and R_{d} represents an alkoxy or alkylamino group.

Yet another exemplary embodiment is directed to a fullerene compound represented by the formula (IV): Cₘ[C(R)NH₂]ₙ, wherein Cₘ, m, n, and R have the same meaning as defined above.

In addition, the disclosure provides a process for producing a fullerene compound represented by the above formula (I), comprising (a) reacting RCH₂CON₃ with a fullerene Cₘ in the presence of a base, to thereby produce the fullerene compound represented by the formula (I), wherein R in RCH₂CON₃ and Cₘ have the same meanings as defined above.

Also described is a process for producing a fullerene compound represented by the above formula (II), comprising heating the fullerene compound represented by the above formula (I).

Further described is a process for producing a fullerene compound represented by the above formula (III), comprising reacting the fullerene compound represented by the above formula (II) with R_{d}H. Alternatively, the fullerene compound represented by the above formula (I) can be heated in the presence of R_{d}H, to produce a fullerene compound represented by the above formula (III).

Moreover, the disclosure provides a process for producing a fullerene compound represented by the above formula (IV), comprising treating the fullerene compound represented by the above formula (III) with an acid.

As described herein, the fullerene compounds described herein, are very useful building blocks, allowing preparation of various functionalized fullerene derivatives.

### BRIEF DESCRIPTION OF THE DRAWINGS

FIGS. 1(a) and 1(b) are partial NMR and FT-IR spectra of an exemplary C₆₀ malonyl azide, respectively.

FIG. 2 is FT-IR spectrum of an exemplary C₆₀ isocyanate.

FIGS. 3(a) and 3(b) are partial NMR and FT-IR spectra of an exemplary t-Boc-protected C₆₀ amine, respectively.

FIG. 4 is NMR spectrum of an exemplary C₆₀ amine.

FIGS. 5(a) and 5(b) are partial NMR and FT-IR spectra of an exemplary C₇₀ malonyl azide, respectively.

FIGS. 6(a) and 6(b) are partial NMR and FT-IR spectra of an exemplary C₇₀ isocyanate, respectively.

FIGS. 7(a)-7(c) are partial NMR, MALDI-MS and FT-IR spectra of an exemplary t-Boc-protected C₇₀ amine, respectively.

FIGS. 8(a) and 8(b) are NMR and FT-IR spectra of an exemplary C₇₀ amine, respectively.

### DETAILED DESCRIPTION OF EMBODIMENTS

The term “methanofullerene,” as used herein, denotes a fullerene derivative comprising at least two methano-bridge atoms, and can be conveniently represented by the formula Cₘ(CR¹R²)ₙ. Cₘ represents an optionally substituted fullerene having m carbon atoms or a derivative thereof. m represents an integer and, in particular, an even value, from about 60 to about 200. In one embodiment, m is 60, 68, 70, 74, 78, 80, 82, 90, 92, and 94. R′ and R² may each represent a monovalent organic group. n is about 1 or more. Theoretically, n is at most m/2. In an embodiment, n is 1. When n is 2 or more, each CR¹R² may be the same or different.

The term “azidating reagent,” as used herein, denotes a compound that can react with an acyl chloride or related compound to form an acyl azide.

The term “acyl chloride related compound,” as used herein, denotes a compound that can, like an acyl chloride, react with an amine to form an amide.

The term “electron withdrawing group,” as used herein, is also referred to as “electron attracting group,” and denotes a group of molecules which exhibits electronegativity, which is a tendency to remove electrons, more than hydrogen.

The term “about,” as used herein, denotes a deviation of ±20% and preferably, ±10%, ±5%, ±2% and ±1%.

U.S. Pat. No. 5,739,376 describes that fullerene undergoes cyclopropanation with an α-halo-CH-acid compound in the presence of a base as follows:

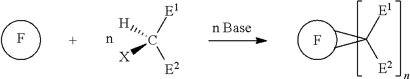

In the above reaction scheme, E¹ and E² can be identical or different and each represent COOH, COOR, CONRR¹, CHO, COR, CN, P(O)(OR)₂, and SO₂R. R and R¹ can each represent a straight-chain or branched, aliphatic radical (C₁ to C₂₀) in which radical up to every third CH₂ unit can be replaced by O or NR⁴. R⁴ is C₁ to C₂₀ alkyl or benzyl. E¹ and E² can also represent an optionally substituted benzyl or phenyl radical. In addition, E¹ and E² can together represent (COO)₂CRR¹. Moreover, E¹ and E² can be different from one another and each represent COR, R or H. Furthermore, E¹ and E² can be different from one another and each represent COR, R, F, Cl, or Br. Additionally, E′ and E² can be different from one another and each represent NO₂, R³ or H. R³ is an unsubstituted, monosubstituted or polysubstituted aliphatic radical (C₁ to C₂₀).

Acyl chlorides and closely related derivatives, such as acyl azides, are known to be very reactive and/or unstable in general, and have not been employed in this type of reactions prior to the present disclosure.

### Preparation of Acyl Azides

As described herein, the acyl azides can be prepared by any suitable methods. In one embodiment, an acylating reagent can be reacted with an azidating reagent. The acylating reagent may be an acyl chloride or acyl chloride related compound. In an embodiment, the acyl chloride has two activated α-H atoms and can be a compound represented by the formula RCH₂COCl, wherein R represents an electron-withdrawing group. The electron-withdrawing group is not particularly limited as long as it is stable and does not interfere with the reaction under the reaction conditions. Examples of suitable electron-withdrawing groups include, but are not limited to, aryl, heteroaryl, alkynyl, halogen, nitro, cyano, P(O)(OR_{b})₂, SO₂R_{b}, and CORₐ. Rₐ represents R_{b}, OR_{b}, or NR_{b}R_{c}. R_{b} and R_{c} each represents H or an optionally substituted straight-chain, branched or cyclic aliphatic group or an optionally substituted aromatic group. The aliphatic and aromatic groups may be substituted by various substituents. Examples of suitable substituents may include, but are not limited to, alkyl, aryl or heteroaryl groups. An aromatic group includes an aryl group containing only carbon and hydrogen atoms, and also a heteroaryl group containing one or more heteroatoms, such as O, N, S, P, etc. Preferably, an aliphatic or aromatic group contains from 1 to 20 carbon atoms.

In addition to acyl chlorides, acyl bromides and the like can also be employed.

An acyl chloride related compound may include, but is not limited to, anhydrides and activated esters having activated α-H atoms. Examples of suitable activated esters include, but are not limited to, compounds having —COOPhCF₃, —COOPhNO₂, and —COOSu. Su represents N-hydroxysuccimide moiety.

The acylating reagent, as described herein, can be prepared by any known methods. For example, acetoacetic acid chloride can be prepared by treating t-butyl acetoacetate with trifluoroacetic acid, followed by treatment of the resultant acid with thionyl chloride.

Examples of suitable azidating reagents may include, but are not limited to, azidotrialkyltin compounds, alkali metal azides, and trialkylsilyl azides. In an embodiment, the alkyl in azidotrialkyltin and trialkylsilyl represents alkyl having 1 to 20 carbon atom and preferably 1 to 8 carbon atoms. Preferably, azidotributyltin is used as the azidating reagent due to its readily availability. Specific examples of suitable alkali metals may include, but are not limited to, sodium and potassium.

In the reaction, the acylating reagent may be used in slight excess to the azidating reagent. In an embodiment, the molar ratio of the acylating reagent and the azidating reagent ranges from about 1.2:1 to about 1:1.

In an exemplary reaction, an acylating reagent, such as an acyl chloride, can be reacted with an azidating reagent, such as azidotributyltin, in an organic solvent or a mixture of two or more organic solvents. The reaction may be conducted in various solvents as long as they do not interfere with the azidation reaction. In general, alkali metal azides are not readily soluble in organic solvents. Therefore, when alkali metal azides are used as the azidating reagent, it is desirable that a phase transferring agent, such as 18-crown-6 and the like, is also employed in the reaction mixture. In a preferred embodiment, the solvent comprises toluene, xylene, benzene, chloro- or bromo-benzene, and/or o-dichlorobenzene. More preferably, the solvent comprises toluene.

The time required for the azidation reaction to be complete depends, at least partially, on the nature of the acylating and azidating reagents employed. In an embodiment, the reaction is substantially complete in about 1 hour to about 4 hours.

The acyl azides thus obtained are found to be relatively stable and can be purified by flash column chromatograph, using, for example, a 1:1 volume mixture of CH₂Cl₂ and hexane and other suitable solvent systems as eluents. It is noted that these acyl azides are moisture sensitive. Therefore, it is preferred that the acyl azides are prepared in situ and used in the next reaction without further purification.

### Preparation of Fullerenyl Acyl Azides Via Cyclopropanation

The cyclopropanation reaction between an acyl azide and a fullerene may be affected by any suitable method. In one embodiment, the reaction conditions described in U.S. Pat. No. 5,739,376 may be employed. In a typical reaction, a fullerene compound can be reacted with an acyl azide in the presence of a base.

The molar ratio of acyl azide and fullerene preferably ranges from about 1.2:1 to about 1:1 in order to obtain a mono-cyclopropanation product of fullerene.

The amount of the base is preferably at least 2 equivalents based on the amount of fullerene. Selection of the base depends, at least partially, on the acidity and reactivity of the α-Hs in the acyl azide. Examples of suitable bases may include, but are not limited to, 1,8-diazabicyclo[5,4,0]undec-7-ene (DBU).

Preferably, a reaction promoter is also employed in the reaction in an amount of about 1 to 2 equivalents based on the amount of fullerene. Examples of suitable promoters include, but are not limited to, iodine, tribromomethane and tetrabromomethane. In an embodiment, the promoter comprises iodine.

This cyclopropanation reaction can be carried out in any organic solvent in which fullerenes have a reasonable solubility and which does not interfere with the cyclopropanation reaction. Examples of suitable organic solvents may include, but are not limited to, toluene, xylene, benzene, chloro- or bromo-benzene, and o-dichlorobenzene. Preferably, the organic solvent comprises toluene.

The cyclopropanation reaction is typically complete in about 1 hour to about 2 hours. The fullerenyl acyl azides are found to be relatively stable, for example, for at least several weeks under dry conditions, and may be purified by flash column chromatograph, using, for example, CS₂ or other suitable solvents as eluent.

Preferably, the acyl azide is freshly prepared prior to the cyclopropanation reaction. In one embodiment, when a freshly prepared malonyl azide was used in the cyclopropanation reaction, the desired product was formed in 68% yield. However, when an aged malonyl azide (e.g., prepared the day before use) was used, 18% of byproduct was collected and the yield of the desired product decreased to 45%.

When multiple cyclopropanation products (i.e., fullerene multi-cyclopropane adducts) are desired, the amounts of acyl azide, iodine and base may be increased proportionally. Furthermore, a fullerene may be treated with two or more different acyl azides, either simultaneously or sequentially, to afford a fullerene having different cyclopropane adducts.

Compared to the synthesis of fullerenyl acyl azide via the diazo addition route described by T. Tada, Y. Ishida, K. Saigo, in The First Synthesis of a Metallo[60] fullerene with an Electron-Donating Group at the Methano-Bridged Carbon: Synthesis and Reaction of Aminomethano[60] fullerene, Org. Lett., 2005, 7, 5897-5900 and Synthesis of 2,2-[60] Fullerenoalkylamines via the Curtius Rearrangement, Synlett, 2007, 2, 235-238, the synthetic route via the malonyl azide described herein has the advantages of convenience in synthesis and diversity of the resulting fullerenyl acyl azides. In addition, the diazo addition has the problem of producing multiple isomers on fullerenes.

### Preparation of Fullerenyl Isocyanates

The fullerenyl acyl azides described herein can be converted into the corresponding fullerenyl isocyanates via any suitable method. In one embodiment, the fullerenyl acyl azides can be transformed into the corresponding fullerenyl isocyanates via Curtius rearrangement. The Curtius rearrangement involves the loss of a N₂ moiety followed by rearrangement of the N, C and O atoms, as illustrated below:

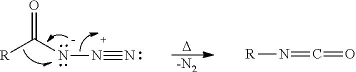

In an exemplary Curtius rearrangement, a fullerenyl acyl azide is heated in an organic solvent at a sufficient temperature and for a sufficient period of time to produce the corresponding fullerenyl isocyanate. Any solvent in which the fullerenyl acyl azides described herein have a reasonable solubility and which does not interfere with the reaction may be employed. Specific examples of suitable solvents may include, but are not limited to, toluene, xylene, benzene and o-dichlorobenzene. Preferably, the organic solvent comprises toluene.

The reaction temperature and time may depend, at least partially, on the nature of fullerenyl acyl azides used. For example, a C₇₀ fullerene derivative generally reacts faster than a C₆₀ fullerene derivative and a fullerene derivative having more than 70 carbon atoms. In an exemplary reaction, the reaction temperature may range from about 60° C. to about 150° C. and preferably, from about 80° C. to about 110° C. The reaction may be substantially complete in about 1 hour to about 16 hours.

The resultant fullerenyl isocyanates may be purified by flash column chromatograph, using, for example, hexane/ethyl acetate, dichloromethane, and ethyl acetate, as eluents. Alternatively, the resultant fullerenyl isocyanates may be used directly in the next reaction without purification.

As described below, the fullerenyl isocyanates described herein can be reactive towards various alcohols and amines under relatively mild conditions, thereby allowing preparation of combinatorial libraries and bioconjugates of new fullerene derivatives.

### Preparation of Fullerenyl Amines

The fullerenyl isocyanates described herein may react with various amines or alcohols represented by the formula R_{d}H to prepare the corresponding carbamides or carbamates. R_{d} represents an oxy or amino group. Amines or alcohols are not particularly limited. Examples of suitable amines and alcohols include, but not limited to, amines and alcohols of optionally substituted linear, branched or cyclic alkanes or aromatics having from 1 to 12 carbon atoms, and preferably from 1 to 6 carbon atoms. For example, n-butyl amine and ethanol react with fullerene isocyanate in very good yields.

Alternatively, the fullerenyl acyl azides described herein may be reacted directly with various amines or alcohols under heating, for example, at a temperature ranging from about 0° C. to about 50° C. and preferably, from about 20° C. to about 25° C., to prepare the corresponding carbamides or carbamates. The reaction conditions may be comparable to those for Curtius rearrangement described herein.

The fullerenyl isocyanates may be reacted with t-butanol to provide tert-butyloxycarbonyl (Boc) protected fullerenyl amines, which may be deprotected to produce free fullerenyl amines where an amino group is attached to the fullerene cage only by a cyclopropane ring.

In an exemplary reaction, a fullerenyl isocyanate is reacted with t-butanol in an organic solvent. T-butanol is typically used in an excess amount, such as about 1.1 equivalents to about 5 equivalents, based on the amount of fullerenyl isocyanate. The reaction may be carried out in a solvent in which the fullerenyl isocyanates described herein have a reasonable solubility and which does not interfere with the desired reaction. Specific examples of suitable solvents may include, but are not limited to, toluene, xylene, benzene and o-dichlorobenzene. Preferably, the solvent comprises toluene.

The reaction time depends, at least partially, on the nature of the fullerenyl isocyanate used. In one embodiment, the reaction is substantially complete in about 1 hour to about 6 hours. The resultant fullerenyl carbamate may be purified by flash column chromatograph, using, for example, ether, dichloromethane, and hexane/ethyl acetate mixtures, as eluents.

The tert-butyloxycarbonyl (Boc) protected fullerenyl amines described herein may be deprotected via any suitable method. In an embodiment, a Boc-protected fullerenyl amine can be treated with a suitable deprotecting agent, such as trifluoroacetic acid (TFA) and the like, in a suitable solvent, such as CH₂Cl₂, to yield the corresponding fullerenyl amine. Moreover, t-Boc-protected fullerenyl amino acid bearing an ethyl ester group on the bridgehead carbon atom may be treated with tribromoborane and TFA, to thereby produce a free acid.

It was reported that aminomethanofullerene bearing an amino group (i.e., an electron-donating group) on the bridgehead carbon can transform to hydrofullerene via a ring-opening reaction. See, e.g., T. Tada, Y. Ishida, K. Saigo, Ring-Opening Reaction of Cyclopropanated [60] Fullerenes: Unexpected Transformation of Methano[60] fullerenes Having an Electron-Donating Group on the Methano-Bridge Carbon, Org. Lett., 2007, 9 (11), 2083-86.

The electron-withdrawing group, such as an ester group, on the bridgehead carbon of the aminomethanofullerenes described herein appears to make the amino compounds more stable.

In fact, these fullerene compounds having an amino acid unit on the bridgehead carbon may play an important role in various applications, including plant biosynthesis. See, e.g., L.-C. Kevin, K. Wang, L. Hai, J. R. Ecker, Ethylene Biosynthesis and Signaling Networks, The Plant Cell, 2002, 14, S131-S151. Furthermore, these fullerene compounds may be directly incorporated into a peptide sequence and may result in compounds having unique biological properties and applications. See, e.g., F. Pellarini, D. Pantarotto, T. Da Ros, A. Giangspero, A. Tossi, M. Prato, A Novel [60] Fullerene Amino Acid for Use in Solid-Phase Peptide Synthesis, Org. Lett., 2001, 3, 1845-48; M. G. Medzhidova, M. V. Abdullaeva, N. E. Fedorova, V. S. Romanova, A. A. Kushch, In Vitro Antiviral Activity of Fullerene Amino Acid Derivatives in Cytomegalovirus Infection, Antibiot. Khimioter, 2004, 49, 13-20.

The fullerene compounds described herein, including fullerenyl malonyl azides, fullerenyl isocyanates, fullerenyl amino acid compounds are very useful building blocks. For instance, functionalization of fullerenes can be achieved simply and conveniently through the coupling between the fullerenyl isocyanates and amines or hydroxyl-containing molecules. The fullerenyl isocyanates can also react with a C-terminal protected amino acid, and the fullerenyl amino acids can react with a N-terminal protected amino acid, thereby providing a convenient path to fullerenyl peptide syntheses.

The present invention is further illustrated by the following specific examples but is not limited hereto.

### EXAMPLES

Unless specified, all the commercially available materials were used herein without further purification. All the measurements including weight and temperature were uncorrected.

### Index of Abbreviations

CDCl₃: deuterotrichloromethane

CH₂Cl₂: dichloromethane

CS₂: carbon disulfide

TEA: triethylamine

Boc: t-butyloxycarbonyl

NMR: Nuclear Magnetic Resonance

J: coupling constant (NMR)

d: doublet (NMR)

t: triplet (NMR)

q: quartet (NMR)

m: multiplier (NMR)

FT-IR: Fourier Transform Infrared

MALDI-MS: Matrix Assisted Laser Desorption/Ionization Mass

### Spectrometry

### Example 1

### Preparation of Malonyl Azide

Ethyl malonyl chloride (0.9 g, 6 mmol) and azidotributyltin (1.99 g, 6 mmol) were stirred in dry toluene (20 mL) overnight. The solution was loaded on a silica gel column and eluted by CH₂Cl₂/hexane 1:1. Rotavaping (100 mbar, 35° C.) offered the desired product as a colorless liquid. Yield was 96%. ¹H-NMR (CDCl₃, 300 MHz, ppm): 4.19 (q, J=7.1 Hz, 2H, CH₂ on the ethyl), 3.35 (s, 2H, CH₂ on the malonate), 1.27 (t, J=7.1 Hz, 3H, CH₃ on the ethyl). ¹³C-NMR (CDCl₃, 75 MHz, ppm): 173.238, 165.727, 61.880, 43.198, 13.998. FT-IR (cm⁻¹): 2140.3, 1742.1, 1712.0.

### Example 2

### Preparation of C₆₀ Malonyl Azide

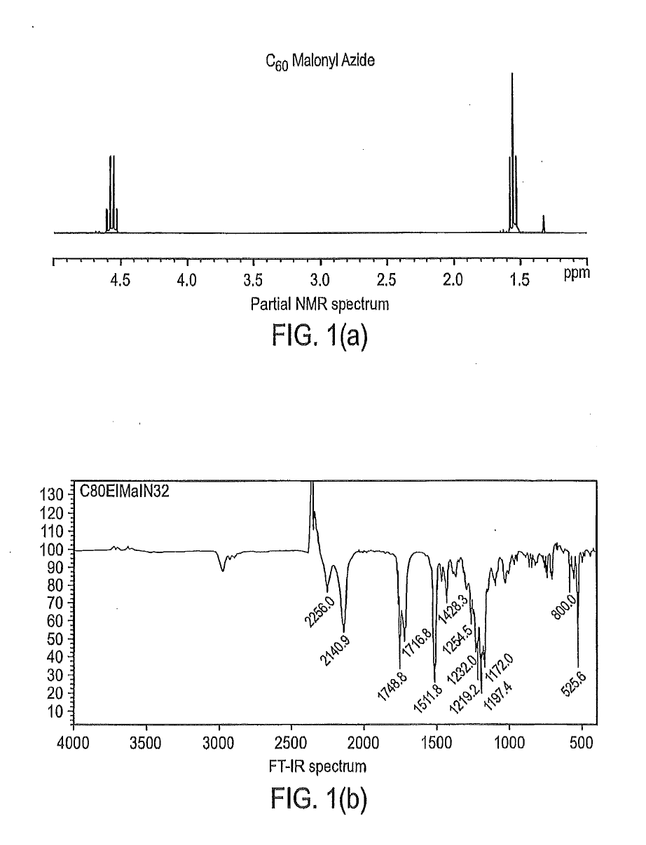

C₆₀ (144 mg, 0.2 mmol), ethyl malonyl azide (31 mg, 0.2 mmol), iodine (56 mg, 0.22 mmol) and 1,8-diazabicyclo[5.4.0]undec-7-ene (DBU, 76 mg, 0.5 mmol) were stirred in dry toluene (150 mL) for 1 hour. Toluene was removed under vacuum, and the residue was diluted with a small amount of CS₂ and loaded to a silica gel column. When eluted by CS₂, the unreacted C₆₀ first came out, and then the desired product was collected as black solid. Yield was 68%. ¹H-NMR (CDCl₃, 300 MHz, ppm): 4.57 (q, J=7.1 Hz, 2H, CH₂), 1.55 (t, J=7.1 Hz, 3H, CH₃) (FIG. 1(a)). ¹³C-NMR (CDCl₃, 75 MHz, ppm): 169.022, 162.126, 145.364, 145.267, 145.124, 145.041, 144.982, 144.851, 144.804, 144.772, 144.707, 144.620, 143.965, 143.952, 143.131, 143.067, 142.316, 141.907, 141.815, 141.147, 141.093, 139.656, 138.841, 71.584, 63.703, 25.677, 14.573. MALDI-MS (m/z): 846 (M⁺-28). FT-IR (cm⁻¹): 2140.9, 1746.8, 1715.8 (FIG. 1(b)).

### Example 3

### Preparation of C₆₀ Isocyanate

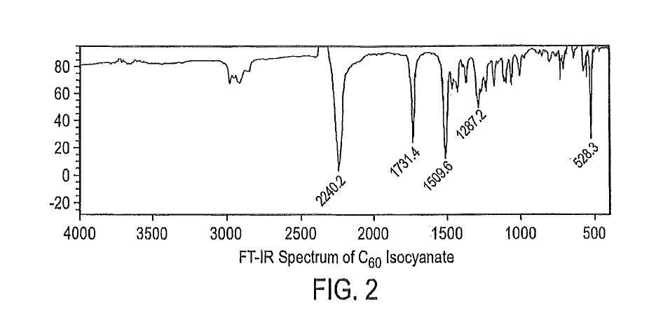

C₆₀ malonyl azide prepared according to Example 2 (87 mg, 0.1 mmol) was heated at 80° C. in dry toluene (100 mL) for 3 hours. Toluene was removed and the black residue was collected as product. Yield was almost quantitative. ¹H-NMR (CDCl₃, 300 MHz, ppm): 4.68 (q, J=7.1 Hz, 2H, CH₂), 1.63 (t, J=7.1 Hz, 3H, CH₃ on the ethyl). MALDI-MS (m/z): 847 (M+H⁺). FT-IR (cm⁻¹): 2240.2, 1731.4 (FIG. 2).

### Example 4

### Preparation of t-Boc Protected C₆₀ Amine

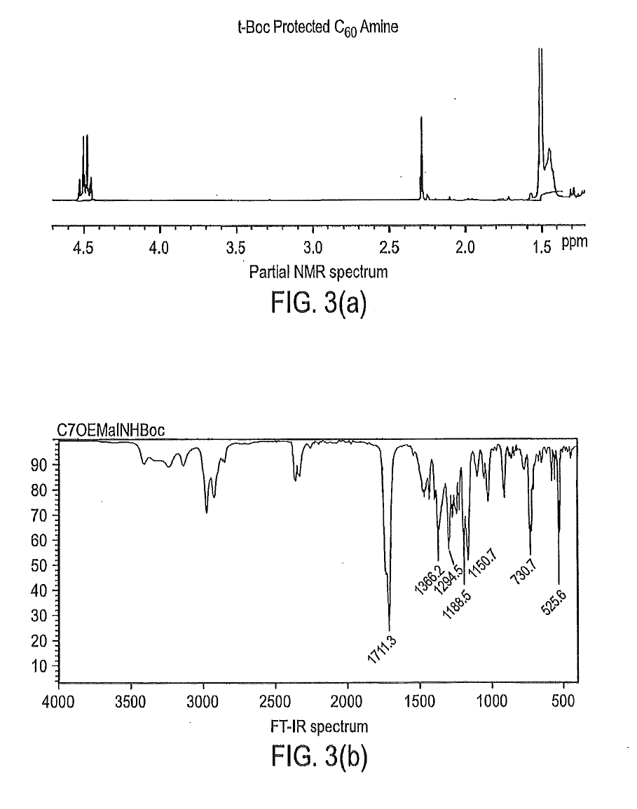

C₆₀ isocyanate prepared according to Example 3 (42 mg, 0.05 mmol) was stirred with t-butanol (370 mg, 5 mmol) in dry toluene (10 mL) overnight. The solution was subjected to silica gel column and eluted by toluene to give t-Boc protected C₆₀ amine. The spectra of t-Boc protected Cₒ amine are shown in FIGS. 3(a) and 3(b).

### Example 5

### Preparation of C₆₀ Amine

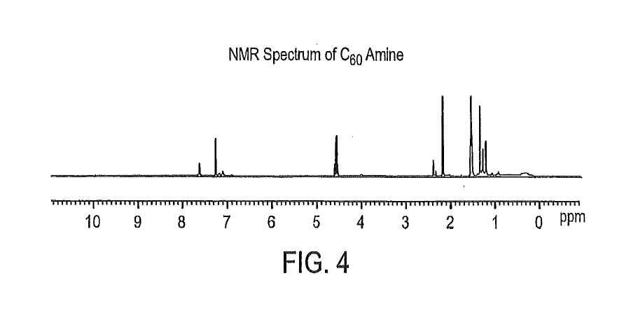

t-Boc protected C₆₀ amine from Example 4 was stirred in CH₂Cl₂ (50 mL) with trifluoroacetic acid (0.5 mL) overnight. After removal of CH₂Cl₂ and trifluoroacetic acid, the residue was re-dissolved into CH₂Cl₂ (50 mL) and stirred with 1 drop of TEA for one hour. The solution was subjected to silica gel column which was eluted by CH₂Cl₂. The yield was 64%. ¹H-NMR (CDCl₃, 300 MHz, ppm): 4.56 (q, J=7.1 Hz, 211, CH₂), 1.53 (t, J=7.1 Hz, 3H, CH₃) (FIG. 4).

MALDI-MS (m/z): 821 (M+H⁺). FT-IR (cm⁻¹): 3281.3, 1744.2.

### Example 6

### Preparation of C₇₀ Malonyl Azide

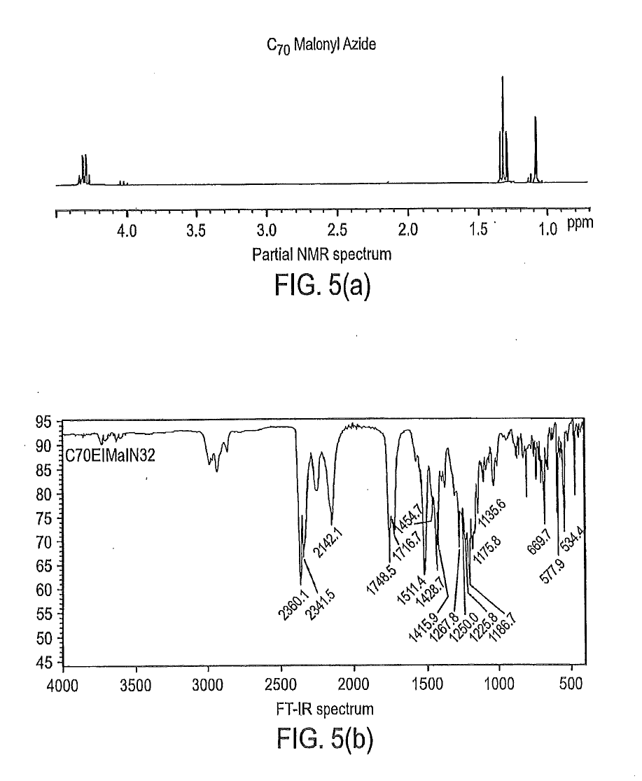

Example 2 was repeated except C₇₀ was used. The C₇₀ malonyl azide was obtained in 63%. ¹H-NMR (CDCl₃, 300 MHz, ppm): 4.53 (doubled q, J=7.1, 1.4 Hz, 2H, CH₂), 1.54 (t, J=7.1 Hz, 3H, CH₃) (FIG. 5(a)). MALDI-MS (m/z): 966 (M⁺-28). FT-IR (cm⁻¹): 2142.1, 1748.5, 1715.7 (FIG. 5(b)). In addition to the C₇₀ malonyl azide, 11% of C₇₀ bisadducts was also collected.

### Example 7

### Preparation of C₇₀ Isocyanate

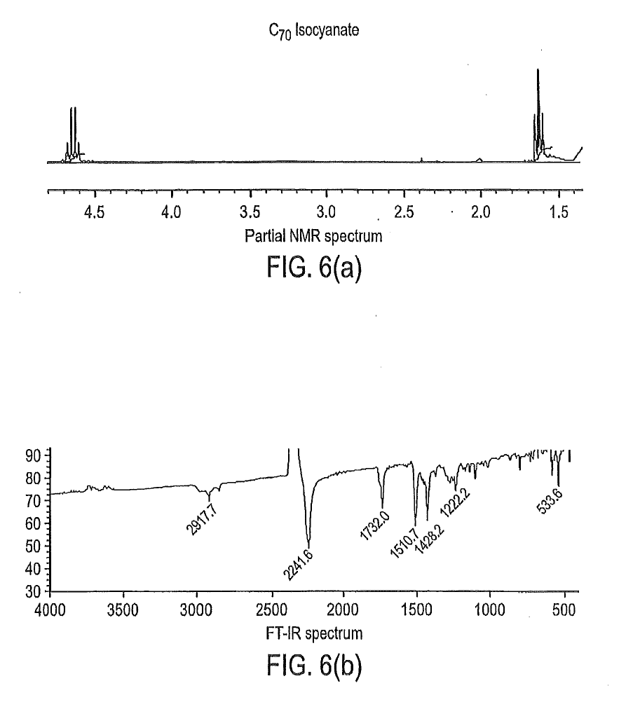

Example 3 was repeated except C₇₀ malonyl azide prepared according to Example 6 was used. ¹H-NMR (CDCl₃, 300 MHz, ppm): 4.60 (q, J=7.1 Hz, 2H, CH₂), 1.61 (t, J=7.1 Hz, 3H, CH₃) (FIG. 6(a)). MALDI-MS (m/z): 967 (M+H⁺). FT-IR (cm⁻¹): 2241.6, 1732.0 (FIG. 6(b)).

### Example 8

### Preparation of t-Boc Protected C₇₀ Amine

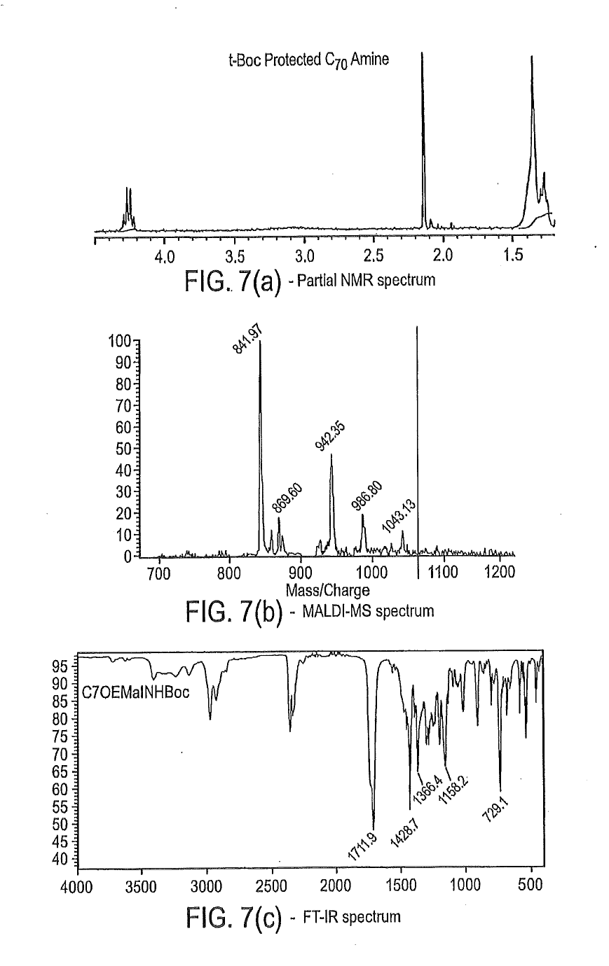

Example 4 was repeated except C₇₀ isocyanate prepared according to Example 7 was used. After purification, the t-Boc protected C₇₀ amine was isolated by chromatography. Spectra of t-Boc protected C₇₀ amine are shown in FIGS. 7(a)-(c).

### Example 9

### Preparation of C₇₀ Amine

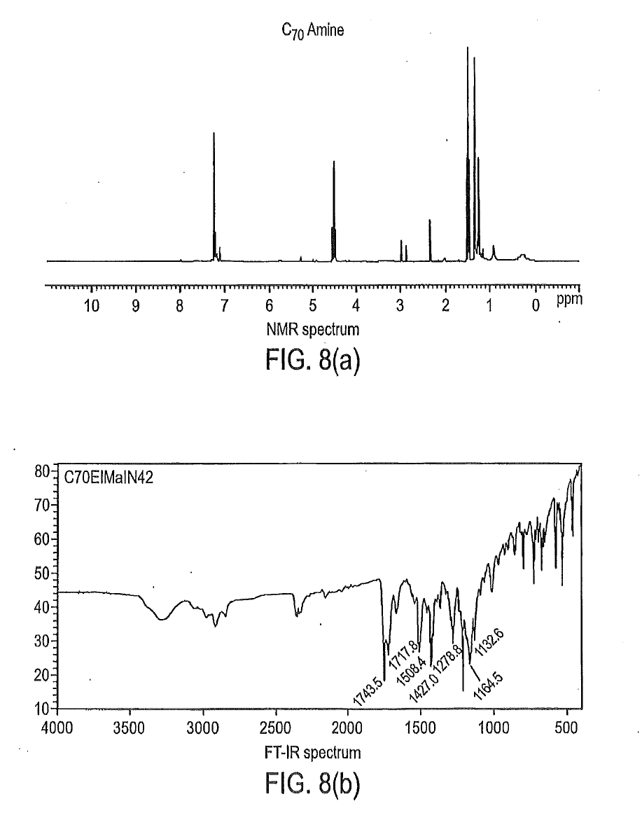

The deprotection of t-Boc protected C₇₀ amine was carried out in the same manner according to Example 5 to give C₇₀ amine. ¹H-NMR (CDCl₃, 300 MHz, ppm): 4.51 (q, J=7.1 Hz, 2H, CH₂), 1.51 (t, J=7.1 Hz, 3H, CH₃) (FIG. 8(a)). MALDI-MS (m/z): 941 (M+H⁺). FT-IR (cm⁻¹): 3277.4, 1743.5 (FIG. 8(b)).

While various embodiments have been described with reference to specific embodiments, variations and modifications may be made without departing from the spirit and the scope of the invention. Such variations and modifications are to be considered within the purview and scope of the invention as defined by the appended claims.

All of the above-mentioned references are herein incorporated by reference in their entirety to the same extent as if each individual reference was specifically and individually indicated to be incorporated herein by reference in its entirety.

1. A fullerene compound represented by the formula (I): Cₘ[C(R)CON₃]ₙ
wherein Cₘ represents a fullerene moiety having m carbon atoms,
m represents an even integer from about 60 to about 200,
n represents an integer of about 1 or more, and
R represents an electron-withdrawing group.

2. The fullerene compound of claim 1, wherein m represents 60, 68, 70, 74, 78, 80, 82, 90, 92, or 94.

3. The fullerene compound of claim 1, wherein n represents 1.

4. The fullerene compound of claim 1, wherein R represents a group selected from the group consisting of aryl, heteroaryl, alkynyl, halogen, nitro, cyano, P(O)(OR_{b})₂, SO₂R_{b}, and CORₐ, wherein Rₐ represents R_{b}, OR_{b}, or NR_{b}R_{c}, and R_{b} and R_{c} each represents H or an optionally substituted straight-chain, branched or cyclic aliphatic group or an optionally substituted aromatic group.

5. A fullerene compound represented by the formula (II): Cₘ[C(R)NCO]ₙ
wherein Cₘ represents a fullerene moiety having m carbon atoms,
m represents an even integer from about 60 to about 200,
n represents an integer of about 1 or more, and
R represents an electron-withdrawing group.

6. The fullerene compound of claim 5, wherein m represents 60, 68, 70, 74, 78, 80, 82, 90, 92, or 94.

7. The fullerene compound of claim 5, wherein n represents 1.

8. The fullerene compound of claim 5, wherein R represents a group selected from the group consisting of aryl, heteroaryl, alkynyl, halogen, nitro, cyano, P(O)(OR_{b})₂, SO₂R_{b}, and CORₐ, wherein Rₐ represents R_{b}, OR_{b}, or NR_{b}R_{c}, and R_{b} and R_{c} each represents H or an optionally substituted straight-chain, branched or cyclic aliphatic group or an optionally substituted aromatic group.

9. A fullerene compound represented by the formula (III): Cₘ[C(R)NHCOR_{d}]ₙ,
wherein Cₘ represents a fullerene moiety having m carbon atoms,
m represents an even integer from about 60 to about 200,
n represents an integer of about 1 or more,
R represents an electron-withdrawing group, and
R_{d} represents an alkoxy or alkylamino group.

10. The fullerene compound of claim 9, wherein m represents 60, 68, 70, 74, 78, 80, 82, 90, 92, or 94.

11. The fullerene compound of claim 9, wherein n represents 1.

12. The fullerene compound of claim 9, wherein R represents a group selected from the group consisting of aryl, heteroaryl, alkynyl, halogen, nitro, cyano, P(O)(OR_{b})₂, SO₂R_{b}, and CORₐ, wherein Rₐ represents R_{b}, OR_{b}, or NR_{b}R_{c}, and R_{b} and R_{c} each represents H or an optionally substituted straight-chain, branched or cyclic aliphatic group or an optionally substituted aromatic group.

13. The fullerene compound of claim 9, wherein R_{d} represents t-butyloxy.

14. A fullerene compound represented by the formula (IV): Cₘ[C(R)NH₂]ₙ,
wherein Cₘ represents a fullerene moiety having m carbon atoms,
m represents an even integer from about 60 to about 200,
n represents an integer of about 1 or more, and
R represents an electron-withdrawing group.

15. The fullerene compound of claim 14, wherein m represents 60, 68, 70, 74, 78, 80, 82, 90, 92, or 94.

16. The fullerene compound of claim 14, wherein n represents 1.

17. The fullerene compound of claim 14, wherein R represents a group selected from the group consisting of aryl, heteroaryl, alkynyl, halogen, nitro, cyano, P(O)(OR_{b})₂, SO₂R_{b}, and CORₐ, wherein Rₐ represents R_{b}, OR_{b}, or NR_{b}R_{c}, and R_{b} and R_{c} each represents H or an optionally substituted straight-chain, branched or cyclic aliphatic group or an optionally substituted aromatic group.

18. The fullerene compound of claim 17, wherein R represents COOR_{b}.

19. A process for producing a fullerene compound represented by the formula (I): Cₘ[C(R)CON₃]ₙ,
wherein Cₘ represents a fullerene moiety having m carbon atoms,
m represents an even integer from about 60 to about 200,
n represents an integer of about 1 or more, and
R represents an electron-withdrawing group,
comprising reacting RCH₂CON₃ with a Cₘ fullerene in the presence of a base, to thereby produce the fullerene compound represented by the formula (I), wherein R in RCH₂CON₃ and Cₘ have the same meanings as defined above.

20. The process of claim 19, further comprising, prior to the reacting, reacting RCH₂COCl with an azidating reagent, thereby obtaining RCH₂CON₃, wherein R in RCH₂COCl has the same meaning as defined in the formula (I).

21. The process of claim 20, wherein the azidating reagent comprises an azidotrialkyltin compound, an alkali metal azide, or a trialkylsilyl azide.

22. The process of claim 21, wherein the azidating reagent comprises an azidotrialkyltin compound.

23. The process of claim 19, wherein m represents 60, 68, 70, 74, 78, 80, 82, 90, 92, or 94.

24. The process of claim 19, wherein n represents 1.

25. The process of claim 19, wherein R represents a group selected from the group consisting of aryl, heteroaryl, alkynyl, halogen, nitro, cyano, P(O)(OR_{b})₂, SO₂R_{b}, and CORₐ, wherein Rₐ represents R_{b}, OR_{b}, or NR_{b}R_{c}, and R_{b} and R_{c} each represents H or an optionally substituted straight-chain, branched or cyclic aliphatic group or an optionally substituted aromatic group.

26. The process of claim 19, wherein the base comprises 1,8-diazabicyclo[5,4,0]undec-7-ene.

27. The process of claim 19, wherein a reaction promoter is added in the reacting (a).

28. The process of claim 27, wherein the reaction promoter comprises I₂.

29. The process of claim 19, wherein the reacting is carried out in toluene.

30-63. (canceled)
